(19) United States
(12) Patent Application Publication
(10) Pub. No.: US 2026/0038660 A1
(43) Pub. Date: Feb. 5, 2026

(54) BEAUTY AND HEALTH INFORMATION PROVIDING SYSTEM, BEAUTY AND HEALTH INFORMATION PROVIDING METHOD, AND BEAUTY AND HEALTH INFORMATION PROVIDING PROGRAM

(71) Applicant: ViewBE Inc., Tokyo (JP)

(72) Inventors: Haruna SUZUKI, Tokyo (JP); Noriaki KOIDE, Tokyo (JP)

(21) Appl. No.: 19/172,213

(22) Filed: Apr. 7, 2025

(30) Foreign Application Priority Data
Apr. 9, 2024 (JP) 2024-062642

(51) Int. Cl. G16H 20/00; G06N 20/00; G16H 30/40; G16H 50/20

(52) U.S. Cl. CPC G16H 20/00; G06N 20/00; G16H 30/40; G16H 50/20

(57) ABSTRACT

[A Problem to be Solved] A technique for easily providing beauty and health information on scalp and/or hair is to be provided.

[Solution] A beauty and health information providing system that provides a user with beauty and health information on scalp and/or hair, the system including: a storage unit that stores a diagnosis model that performs learning using inquiry information on an answer from a user to an inquiry and a captured image of the scalp and/or hair as input data and using a graded evaluation of the scalp and/or hair as output data; a reception unit that receives the inquiry information on the answer from the user to the inquiry and the captured image of the scalp and/or hair; and a diagnosis unit that determines a diagnosis result that is the graded evaluation of the scalp and/or hair based on the inquiry information and the captured image by using the diagnosis model.

### CROSS REFERENCE TO RELATED APPLICATIONS

This application claims the benefit of foreign priority to Japanese Patent Application No. 2024-062642, filed on Apr. 9, 2024, which is incorporated by reference in its entirety.

### TECHNICAL FIELD

The present invention relates to a beauty and health information providing system, a beauty and health information providing method, and a beauty and health information providing program.

### BACKGROUND ART

Recently, a user can easily receive advice on his or her health from an expert or the like by providing information on the user using a terminal such as a smartphone.

Patent Literature 1 discloses a skin condition determination system that outputs a result of skin diagnosis by providing an inquiry content performed using a mark sheet or the like and image data of sebum.

### CITATION LIST

### Patent Literature

Patent Literature 1: JP2016-077649A

### SUMMARY OF INVENTION

### Technical Problem

However, in the determination system described above, an image of keratin or sebum acquired by a sampling device using an adhesive substance such as a keratin checker or a sebum checker is used, but the sampling device using such an adhesive substance cannot be used for hair or scalp, and an expensive diagnosis instrument is required to diagnose hair or scalp, so that it is not possible to easily provide a diagnosis result for hair or scalp.

In view of the above problem, an object of the present invention is to provide a technique for easily providing beauty and health information on scalp and/or hair.

### Solution to Problem

In order to solve the above problem, the present invention provides
a beauty and health information providing system that provides a user with beauty and health information on scalp and/or hair, the system including:
a storage unit that stores a diagnosis model that performs learning using inquiry information on an answer from a user to an inquiry and a captured image of the scalp and/or hair as input data and using a graded evaluation of the scalp and/or hair as output data;
a reception unit that receives the inquiry information on the answer from the user to the inquiry and the captured image of the scalp and/or hair; and
a diagnosis unit determines a diagnosis result that is the graded evaluation of the scalp and/or hair based on the inquiry information and the captured image by using the diagnosis model.

With such a configuration, it is possible to easily provide an inquiry result, which is the graded evaluation, based on simple data including the answer to the inquiry and image data.

In a preferred embodiment of the present invention, the beauty and health information providing system further includes a learning unit, in which
the learning unit performs learning of the diagnosis model by using the inquiry information on the answer from the user to the inquiry and the captured image of the scalp and/or hair as input data and using the graded evaluation of the scalp and/or hair as output data.

With such a configuration, it is possible to perform the learning of the diagnosis model capable of performing the graded evaluation.

In a preferred embodiment of the present invention, the storage unit stores history information that is a past diagnosis result of the user, and
the diagnosis unit determines the diagnosis result further based on the history information.

With such a configuration, it is possible to provide a diagnosis result in consideration of the past diagnosis result.

In a preferred embodiment of the present invention, the beauty and health information providing system further includes a display processing unit, in which
the diagnosis result includes graded evaluations of a plurality of evaluation items for the scalp and/or hair, and
the display processing unit performs display processing of a diagnosis result display screen that displays the graded evaluations of the plurality of evaluation items for the scalp and/or hair based on the diagnosis result.

With such a configuration, it is possible to display the graded evaluations related to the plurality of evaluation items and easily provide information on the diagnosis result to the user.

In a preferred embodiment of the present invention, the diagnosis unit stores the history information that is the past diagnosis result of the user in the storage unit in association with a diagnosis date, and
the diagnosis result display screen includes a progress display diagram showing evaluation result progress for the scalp and/or hair based on a plurality of the diagnosis results and the diagnosis dates.

With such a configuration, it is possible to provide the evaluation progress in a form that is easy for the user to intuitively understand based on a plurality of diagnosis results such as the past diagnosis results.

In a preferred embodiment of the present invention, the beauty and health information providing system further includes an advice providing unit, in which
the advice providing unit outputs advice on beauty care of the scalp and/or hair based on the inquiry information.

With such a configuration, it is possible to provide advice such as a care method based on the diagnosis result to the user.

In a preferred embodiment of the present invention, the beauty and health information providing system further includes a care information providing unit, in which
the storage unit further stores a plurality of pieces of care information on care to be provided to the user, and
the care information providing unit provides the care information on care suitable for the user based on the diagnosis result and the plurality of pieces of care information.

With such a configuration, it is possible to provide information on care such as a product suitable for the user based on the diagnosis result.

### Advantageous Effects of Invention

The present invention can provide a novel technique for providing beauty and health information on the scalp and/or hair.

### BRIEF DESCRIPTION OF DRAWINGS

FIG. 1 is a block diagram illustrating a configuration of an entire system.

FIG. 2 is a hardware configuration diagram of the system.

FIG. 3 illustrates a data configuration and an example of a captured image.

FIG. 4 illustrates an example of screen display.

FIG. 5 illustrates an example of screen display.

FIG. 6 is a flowchart illustrating a processing flow.

### DESCRIPTION OF EMBODIMENTS

Hereinafter, a beauty and health information providing system, a beauty and health information providing method, and a beauty and health information providing program of the present invention will be described with reference to the drawings. Note that an embodiment described below is an example of the present invention, and the present invention is not limited to the embodiment described below, and various configurations can be adopted.

In the present embodiment, a configuration, operations, and the like of the beauty and health information providing system will be described, but a method, a device, a computer program, a program storage medium storing the program, and the like having the same configuration can achieve the same functions and effects. A series of processing according to the present embodiment described below is provided as a program executable by a computer, and can be provided through a non-transitory computer readable recording medium such as a CD-ROM or a flexible disk, or a communication line.

### System Configuration

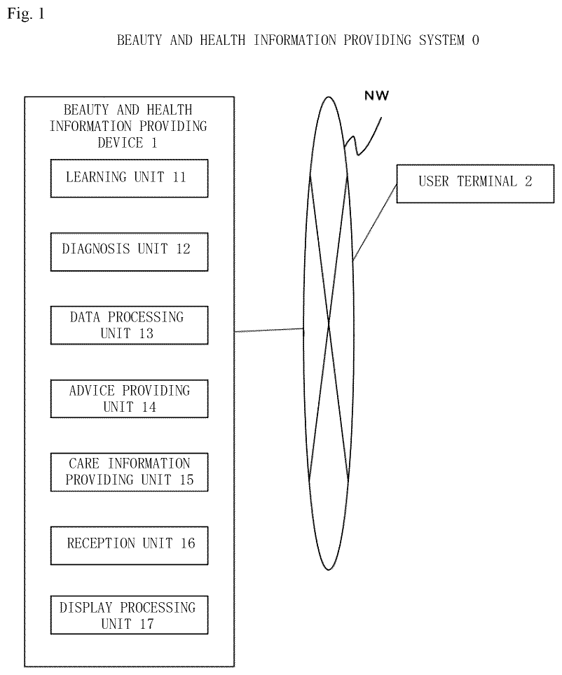

FIG. 1 is a system configuration diagram of a beauty and health information providing system 0. As illustrated in FIG. 1, the beauty and health information providing system 0 includes a beauty and health information providing device 1 and a user terminal 2, and each component is communicably connected via a communication network NW. The communication network NW is any network using any communication protocol, such as the Internet Protocol (IP) network. In the present embodiment, the beauty and health information providing system 0 may further include a provider terminal 3 that is a terminal device of a provider such as an expert who provides information on beauty care to a user.

The user terminal 2 is a terminal device used by the user who receives beauty and health information on scalp and/or hair using the beauty and health information providing system. Although only one user terminal 2 is illustrated in FIG. 1, there are a plurality of user terminals 2 in the present embodiment. In the present embodiment, the user terminal 2 receives input of necessary information such as information on the scalp and/or hair from the user including inquiry information and a captured image, and transmits the information to the beauty and health information providing device 1.

In the present embodiment, the user inputs an answer to the inquiry and captures an image of the scalp and/or hair via the user terminal 2 owned by the user, and transmits information on the scalp and/or hair of the user such as a content of the inquiry and the captured image to the beauty and health information providing device 1, thereby receiving the beauty and health information such as a diagnosis result related to the scalp and/or hair. In the present embodiment, the user can further receive, from the beauty and health information providing device 1, advice according to his or her trouble or diagnosis result and product information on a product suitable for the user as the beauty and health information. The beauty and health information provided by the beauty and health information providing system 0 is information on beauty and/or health, and is information on beauty care such as a diagnosis result, advice information, and care information in the present embodiment.

### Hardware Configuration

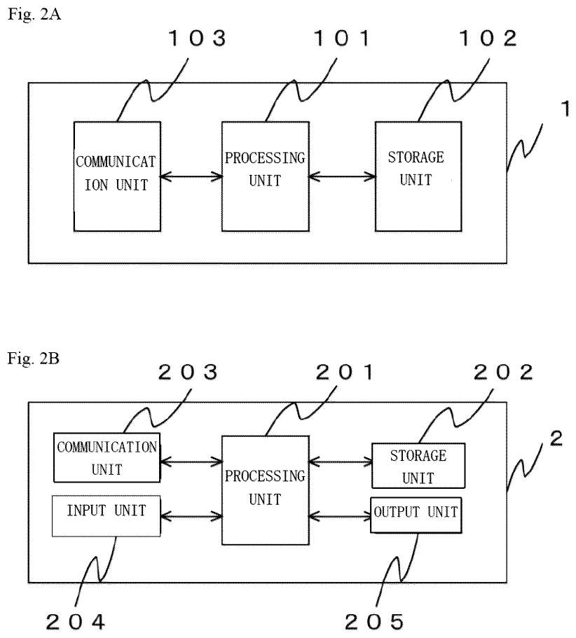

“(a)” of FIG. 2 is a hardware configuration diagram of the beauty and health information providing device 1. The beauty and health information providing device 1 includes, as a hardware configuration, a processing unit 201, a storage unit 202, and a communication unit 203. In the present embodiment, a computer device such as a server or a personal computer can be used as the beauty and health information providing device 1. Note that the beauty and health information providing device 1 may include a plurality of computer devices, and is not limited to the configuration illustrated in “(a)” of FIG. 2 as long as functional components (11 to 17) described below can be implemented as a whole.

The processing unit 101 includes one or more processors such as a CPU, and controls overall processing in the beauty and health information providing device 1 by executing the beauty and health information providing program, an operating system (OS), and other applications. The storage unit 102 includes one or more memories such as a hard disk drive (HDD), a solid state drive (SSD), a flash memory, or a random access memory (RAM), and stores the beauty and health information providing program and various kinds of data. The communication unit 103 controls communication with the communication network NW and achieves data communication with the user terminal 2 and the like. The processing unit 101 executes the beauty and health information providing program to cause the computer to function as the beauty and health information providing device 1 and execute the beauty and health information providing method.

In the present embodiment, the storage unit 102 of the beauty and health information providing device 1 stores data used as teaching data including the inquiry information, the captured image, and evaluation information, and various necessary information such as a model and user information used in the processing of the system including an evaluation model.

“(a)” of FIG. 2 is a hardware configuration diagram of the user terminal 2. The user terminal 2 includes a processing unit 201, a storage unit 202, a communication unit 203, an input unit 204, and an output unit 205 as a hardware configuration. In the present embodiment, a terminal device such as a smartphone, a personal computer, or a tablet terminal can be used as the user terminal 2. A terminal device used as the provider terminal 3 that provides the beauty and health information to the user may have a hardware configuration similar to that of the user terminal 2 as shown in “(b)” of FIG. 2.

The processing unit 201 includes one or more processors such as a CPU, and controls overall processing in the user terminal 2 by executing an OS and other applications. The storage unit 202 is an HDD, an SSD, a flash memory, a RAM, or the like, and stores a browser application and various kinds of data. The communication unit 203 controls communication with the communication network NW and achieves data communication with the beauty and health information providing device 1 and the like. The input unit 204 is an input interface that receives an operation request or the like from an operator, and includes a touch panel, a mouse, a keyboard, and the like. The output unit 205 includes a display or the like that displays a processing result by the processing unit 201.

The beauty and health information providing device 1 stores the diagnosis model in the storage unit 102, and inputs the inquiry information on the answer from the user to the inquiry and the captured image of the scalp and/or hair to the diagnosis model to determine the diagnosis result that is the graded evaluation of the scalp and/or hair.

### Diagnosis Model

The beauty and health information providing device 1 stores in the storage unit 102 the diagnosis model that performs learning by the learning unit 11, and the diagnosis unit 12 determines the diagnosis result using this diagnosis model. Hereinafter, the diagnosis model in the present embodiment will be described.

The diagnosis model is a learned model obtained by learning using, as input data, information on the hair of the user including the inquiry information on the answer from the user to the inquiry and the captured image of the scalp and/or hair of the user, and using, as output data, the graded evaluation of the scalp and/or hair determined by an expert in scalp, hair, and the like based on the answer to the inquiry and the captured image. In the present embodiment, the diagnosis model learns the evaluation performed by the expert based on the captured image of the scalp and/or hair, the inquiry information on the content of the inquiry from the user, and evaluation information on the graded evaluation performed by the expert who has confirmed the captured image and the content of the inquiry from the user. In this way, by performing learning based on the information on the user confirmed by the expert including the captured image and the inquiry information and the evaluation performed by the expert, the diagnosis model in the present application can determine a graded diagnosis result similar to that provided by an expert based on the inquiry information and the captured image, instead of a diagnosis result based on strict numerical values. In the present embodiment, when the inquiry information and the captured image are input, the diagnosis model determines the diagnosis result that is the graded evaluation such as a five-grade evaluation with respect to a plurality of evaluation items such as fineness of hair, a moisture content of hair, and an amount of hair.

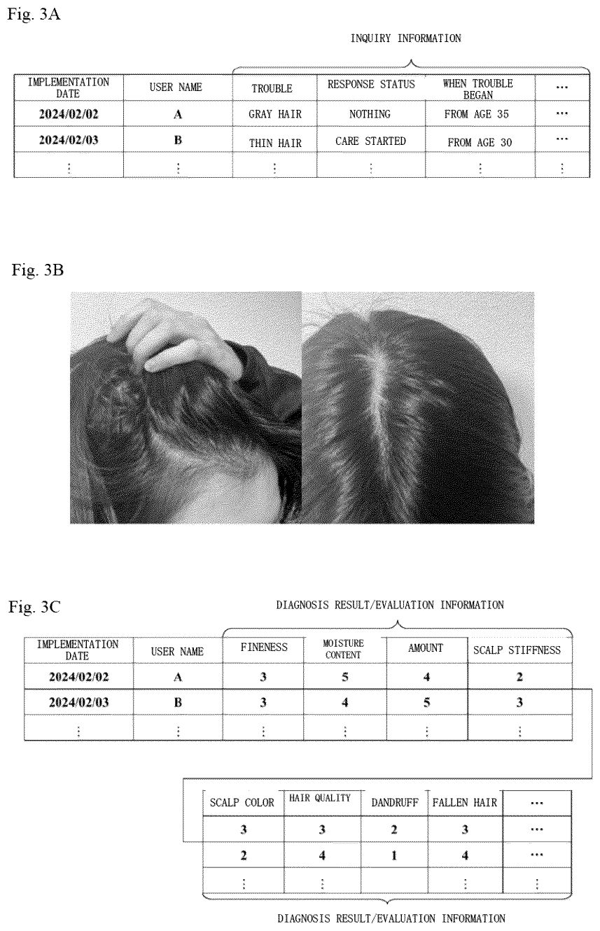

An example of data used for learning of the diagnosis model and determining of the diagnosis result using the diagnosis model will be described below with reference to FIG. 3. A data configuration described below is an example in the present embodiment, and the present invention is not limited to the data configuration illustrated in the drawing. The inquiry information, the captured image, and the diagnosis result/evaluation information related to the same diagnosis are stored in the storage unit 102 in association with each other using identification information such as an ID.

“(c)” of FIG. 3 illustrates an example of the inquiry information. The inquiry information is information on the answer from the user to the inquiry input by the user terminal 2. In the present embodiment, the inquiry information is an answer from the user to questions about the scalp and/or hair of the user, such as a trouble related to the scalp or hair of the user, a response status to the trouble of the user, and a time when the trouble of the user starts, and is acquired as text data for receiving an input other than options. In addition, the inquiry information may be any information as long as it is information that can be used for diagnosis of the scalp and/or hair, and may be, for example, information on the user himself or herself such as age and gender of the user, information on a care product that the user uses for care of the scalp and/or hair, and information on the state of the scalp and/or hair of the user considered by the user himself or herself. The inquiry information may include information on the question asked to the user and information on the answer from the user in association with each other.

“(b)” of FIG. 3 illustrates an example of the captured image of the scalp and/or hair used for learning of the diagnosis model and processing related to the determination of the diagnosis result. In the present embodiment, the captured image is an image related to the hair or the scalp of the user captured by the user terminal 2, and is an image acquired by imaging a head of the user from a plurality of designated angles. The captured image may be a plurality of pieces of image data, or may be an image obtained by combining images captured from a plurality of angles.

“(c)” of FIG. 3 illustrates an example of a data configuration of the diagnosis result and the evaluation information. In the present embodiment, the diagnosis result and the evaluation information have the same data configuration.

The diagnosis result is a result of diagnosis of the scalp and/or hair of the user determined using the diagnosis model based on the inquiry information and the captured image, and is a graded evaluation (such as a five-grade evaluation) of evaluation items such as the fineness of the hair, the moisture content of the hair, the amount of the hair, stiffness of the scalp, and color of the scalp. In the present embodiment, the diagnosis result includes graded evaluations of a plurality of evaluation items.

The evaluation information is information on the graded evaluations related to the plurality of evaluation items performed by an expert such as a hair diagnostician or a doctor who has confirmed the captured image related to the scalp and/or hair and the answer from the user to the inquiry, which is used as teaching data for performing learning of the diagnosis model. The evaluation information is information on the graded evaluations (such as five-grade evaluations) of the evaluation items such as the fineness of hair, the moisture content of hair, the amount of hair, the stiffness of scalp, the color of scalp, and the like, and includes the graded evaluation of each evaluation item. In the present embodiment, the diagnosis model determines a graded evaluation as the diagnosis result for the same evaluation item as the item in the evaluation information used as the teaching data.

The diagnosis model may determine the diagnosis result based on a past diagnosis result of the user in addition to the captured image and the inquiry result. In the present embodiment, the diagnosis model is a model common to any user and trouble, but may be a model suitable for each user, such as the age and gender of the user and the trouble of the user, selected based on the user information and/or the inquiry information.

The diagnosis model in the present embodiment is a multimodal model configured by combining models capable of processing natural language and image data, such as receiving, as input data, data generated by the data processing unit 13 preprocessing the captured image as the image data and the inquiry information as the text data. The diagnosis model may be a model configured by using a plurality of models different from each other such that, in a case where each piece of data of the captured image and the inquiry information is input to a model that performs image processing and a different model that performs natural language processing, each model determines a diagnosis result based on a feature extracted from the captured image and the inquiry information, respectively. In the present embodiment, the diagnosis model is a model configured by a neural network such as a convolutional neural network. In a case where the reception unit 16 receives one answer selected from a plurality of options in the inquiry as the inquiry information, the diagnosis model may be a model that cannot perform natural language processing, such as performing supervised learning based on the answer selected from the options acquired as the inquiry information and the evaluation by the expert and determining the diagnosis result (a value of the graded evaluation).

In addition, the diagnosis model in the present embodiment is a multi-input multi-output model that receives, as inputs, a plurality of pieces of data including a vector of a feature of an image acquired as the captured image and a vector acquired by vectorizing a sentence acquired as the inquiry information, and outputs evaluations for the plurality of evaluation items as the diagnosis result. As described above, in the diagnosis model according to the present embodiment, the captured image as the image data and the inquiry information as the text data are converted into vectors and handled by one model, and then the processing related to the determination of the diagnosis result is performed, so that a processing speed is improved as compared with a case where a plurality of models corresponding to individual inputs are generated to determine the diagnosis result. The diagnosis model is a supervised model in which learning is performed by attaching the evaluation from the expert acquired as the evaluation information as a label to the vector of the feature of the image acquired as the captured image and the vector of the sentence acquired as the inquiry information.

In the present embodiment, the diagnosis model is configured by combining a convolutional neural network for determining the diagnosis result and a large-scale language model for generating advice, and also functions as an advice providing model. The diagnosis model and the advice providing model may be configured as separate models.

### Functional Configuration

The beauty and health information providing device 1 is a device for providing beauty and health information such as the diagnosis result of the scalp and/or hair, and includes the learning unit 11, the diagnosis unit 12, the data processing unit 13, an advice providing unit 14, a care information providing unit 15, the reception unit 16, and a display processing unit 17. Hereinafter, a functional configuration of the beauty and health information providing device 1 will be described.

The learning unit 11 performs the learning of the diagnosis model using the inquiry information on the answer from the user to the inquiry and the captured image of the scalp and/or hair as the input data and the graded evaluation of the scalp and/or hair as the output data. In FIG. 1, the learning unit 11 is included in the beauty and health information providing device 1, but may be included in another server device used as a learning device. The learning unit 11 may perform processing of adjusting a learned model, such as performing fine-tuning using additionally acquired data. Note that the learned diagnosis model that performs learning by the learning unit 11 is stored in the storage unit 102 of the beauty and health information providing device 1, and is used for processing related to the determination of the diagnosis result in the diagnosis unit 12 described later. In the present embodiment, the learning unit 11 performs the learning using data after processing (processed data) on which preprocessing for conversion into a form that is easy to handle by the data processing unit 13 has been performed, but in a case where processing is unnecessary for data (raw data) on which preprocessing has not been performed, learning may be performed using raw data.

The reception unit 16 receives information on the scalp and/or hair of the user including the inquiry information and the captured image from the user terminal 2, and performs processing of delivering the information to the diagnosis unit 12 or the like. The reception unit 16 may store the received information including the inquiry information in the storage unit 102 for use in processing in the beauty and health information providing device 1 such as the determination of the diagnosis result. In addition, the reception unit 16 may receive the input of the beauty and health information provided to the user and the information (evaluation information) on the graded evaluation of the scalp and/or hair used as the teaching data, which are input by the provider terminal 3 operated by a provider such as an expert.

The data processing unit 13 performs, as preprocessing, processing of converting various kinds of data such as teaching data including the inquiry information, the captured image, and the evaluation information and data used for diagnosis including the inquiry information and the captured image received from the user terminal 2 into a form easy to handle in the beauty and health information providing system 0. Although the data that has not been subjected to the preprocessing is referred to as raw data and the data that has been subjected to the preprocessing is referred to as processed data, the data processing unit 13 may not perform the preprocessing when the acquired raw data has a format that is easily to be handled by the beauty and health information providing system 0.

The data processing unit 13 may perform preprocessing on the captured image by performing processing of converting a color image into a monochrome image, processing of combining a plurality of pieces of image data captured from a plurality of angles into one piece of image data by combining the plurality of pieces of image data in left and right as in the example illustrated in FIG. 3, or the like. The data processing unit 13 may perform preprocessing on the inquiry information acquired as text data by performing processing of vectorizing a sentence or converting a sentence into a token. The data processing unit 13 may perform the same processing as the processing on the captured image and the inquiry information as preprocessing for other image data and text data.

Based on the inquiry information and the captured image received by the reception unit 16 from the user terminal 2, the diagnosis unit 12 determines the diagnosis result that is the graded evaluation of the scalp and/or hair, using the diagnosis model. In the present embodiment, the diagnosis result determined by the diagnosis unit 12 includes the graded evaluations of the plurality of evaluation items such as the fineness of hair and the moisture content of hair, similar to the items in the evaluation information used for learning as teaching data.

In the present embodiment, the diagnosis unit 12 stores the determined diagnosis result in the storage unit 102 in association with user information of a user to be diagnosed and information on the date and time when the diagnosis was performed, such as a diagnosis date, for use in processing of providing the information on progress of the diagnosis result to the user. The storage unit 102 stores the diagnosis result in the diagnosis performed by the user in the past as history information, but may further store the inquiry information and the captured image used for determining the diagnosis result in association with the diagnosis result. Further, when the user has been diagnosed in the past, the diagnosis unit 12 determines the diagnosis result referring to the past diagnosis result of the user based on the past diagnosis result (history information) of the user stored in association with the user information in addition to the captured image and the inquiry information.

The advice providing unit 14 outputs advice based on the diagnosis result from the diagnosis unit 12. In the present embodiment, as processing of outputting advice, the advice providing unit 14 performs processing of generating advice using an advice providing model, which is a language model that performs learning based on text data such as advice provided by an expert or interaction between the user and the expert in a chat. In the present embodiment, the advice providing model is a model capable of generating advice on the scalp and/or hair to the user, which performs learning using the inquiry information and the captured image, and information (advice history) on advice provided in the past by an expert after checking the inquiry information and the captured image. The advice providing model in the present embodiment is a large-scale language model capable of generating a sentence, and is a model that outputs advice when a vector of an image acquired as the captured image and a vector obtained by vectorizing a sentence acquired as the inquiry result is input similarly to the diagnosis model, but may further receive the past diagnosis result (diagnosis history) as an input. The advice providing unit 14 may generate a prompt to be input to the advice providing model based on the inquiry information, the captured image, and the diagnosis result, and output advice generated using the generated prompt and the advice providing model.

The advice providing model may be a model that determines which advice is to be provided based on the inquiry information, the captured image, and the diagnosis result. In this case, the advice providing unit 14 determines a classification (advice classification) regarding the advice to be provided based on the inquiry information, the captured image, and the diagnosis result, and outputs advice corresponding to the determined classification with reference to advice information stored in the storage unit 102. The advice information stored in the storage unit 102 is information on advice set for each classification (advice classification) determined by the advice providing model, and includes information on the classification by the advice providing model and a content of the advice corresponding to the classification (for example, when a classification of premature gray hair is determined, advice with a content such as “It is good to take care of premature gray hair with . . .” corresponding to the classification of premature gray hair). For example, when the trouble of the user is the fineness of hair and the evaluation of the item of the fineness of hair in the diagnosis result is 1 (1 is the thinnest and 5 is the thickest) in the five-grade evaluation, the advice providing unit 14 refers to the advice information and outputs advice corresponding to the classification determined by the advice providing model, such as advice of “In the case of thin hair, it is preferable to take care such as . . .”.

The care information providing unit 15 determines a care suitable for the user, such as a product suitable for the user corresponding to a hair quality or the like of the user, based on the care information on a plurality of cares stored in the storage unit 102 and the diagnosis result, and provides care information on the determined care. The care information stored in the storage unit 102 is information on care to be provided to the user, including product information on products used for the care of the scalp and/or hair, such as hair care products and scalp care products, component information on beauty care components and the like that should be taken by the user, training information on training that should be performed by the user, recipe information on foods that should be taken by the user, and consultation destination information on experts (for example, a dietician, a beautician, a doctor, and the like) who should be consulted for hair. In the present embodiment, for example, when a result indicating that the hair is thin appears as the diagnosis result, the care information providing unit 15 determines a care suitable for the user based on the diagnosis result and provides care information on the determined care, such as providing information on a care for a person with thin hair. In this case, the care information providing unit 15 may classify the user based on the diagnosis result and provide the care information for each classification based on the classification, may provide information on the care related to the item of which an evaluation farthest from an average is obtained in the diagnosis result of the user (for example, in a case where evaluations of other items are on an average level, but only the evaluation result for the item of the fineness of hair is far from the average, the care information providing unit 15 may provide the care information on the care of countermeasure against the thin hair, or the like), or may provide the care information on the item designated by the user (in a case where the item related to the fineness of hair is selected, the care information providing unit 15 may provide the information on the care according to the fineness of hair of the user obtained from the diagnosis result, or the like).

Hereinafter, regarding the provision of the care information, an example in which the product information on the product suitable for the user is provided will be described. In the present embodiment, the product information includes a product name, a product image, a product ID, and a provided product classification. In the present embodiment, the care information providing unit 15 determines a provided product classification, which is a classification of a product whose information is provided to the user, based on the diagnosis result, and provides the product information on the product belonging to the determined classification based on the provided product classification and the product information. The care information providing unit 15 may determine the product whose information is to be provided, such as a product suitable for the trouble of the user, based on other information used for diagnosis such as the inquiry information and the captured image. In this case, the care information providing unit 15 performs classification regarding the product whose information is provided based on any one or more of the diagnoses results, the inquiry information, and the captured image, and provides the product information suitable for the user, based on the determined provided product classification, with reference to the product information on the product belonging to the classification. In addition, in a case where a plurality of products are registered with respect to the determined provided product classification, the care information providing unit 15 may determine a product whose product information is to be provided from among the products registered in the provided product classification based on information on the user such as an age and a gender, or may provide information on the plurality of products corresponding to the determined provided product classification.

The care information providing unit 15 may provide product information on a specific product in a case where the evaluation of any one or a plurality of evaluation items is within a predetermined range based on the diagnosis result, such as providing information on a product for a person with thin hair in a case where the evaluation of the fineness of hair is 1 to 2 (1 is the thinnest and 5 is the thickest), based on the evaluation range of the predetermined evaluation item included in the product information and the evaluation of the predetermined evaluation item included in the diagnosis result.

The display processing unit 17 performs display processing of a screen including a diagnosis result display screen based on various kinds of necessary information including the diagnosis result. Hereinafter, a screen displayed by the display processing unit 17 will be described with reference to the drawings. In the present embodiment, the display processing unit 17 displays, on the user terminal 2, the diagnosis result and the beauty and health information including the advice and the care information provided by the advice providing unit 14, so that the beauty and health information providing system 0 provides the beauty and health information to the user.

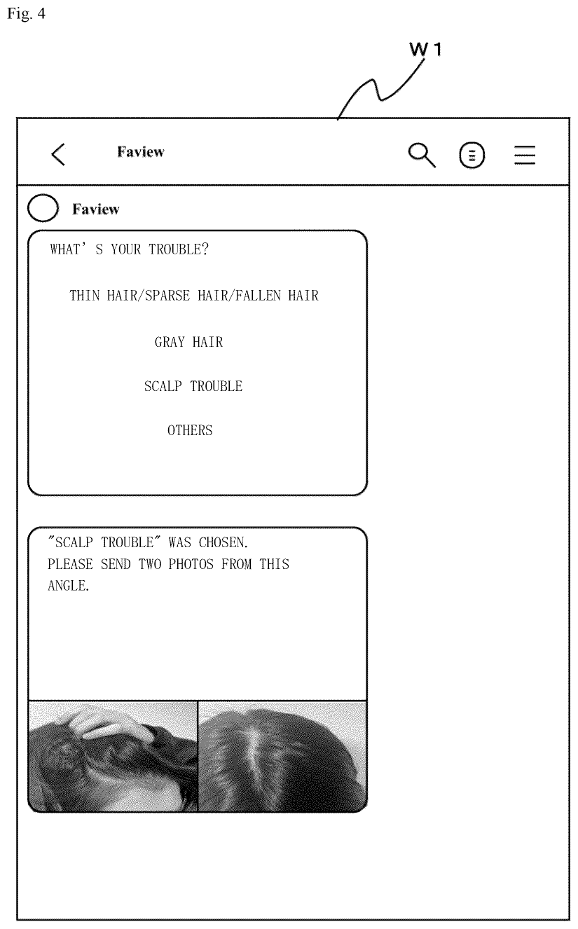

FIG. 4 illustrates an example of a screen display of an information input screen which is displayed on the user terminal 2 when the user inputs information necessary for performing diagnosis including the inquiry information and the captured image. An information input screen W1 illustrated in the drawing is a screen for receiving input of various kinds of information necessary for diagnosing the scalp and/or hair including the inquiry information and the captured image, which is displayed when the reception unit 16 receives a selection of starting diagnosis from the user. In the present embodiment, the display processing unit 17 displays a question on the information input screen W1 based on question information stored in the storage unit 102, and the reception unit 16 receives an answer to the displayed question as the inquiry information. In this case, the display processing unit 17 may determine the question to be displayed based on characteristics of the user such as the age and the gender based on the user information and answers from the user to other questions such as the content of the trouble of the user, and perform the question display processing. In this case, the reception unit 16 may receive inquiry information that varies depending on the gender, age, or the like of the user (for example, whether the user is pregnant or after birth in the case of a female). The reception unit 16 receives the inquiry information and the captured image input on the information input screen W1.

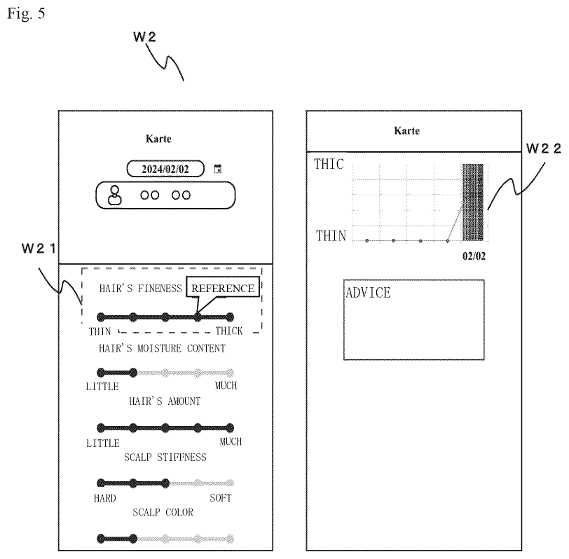

FIG. 5 illustrates an example of a screen display of a diagnosis result display screen that displays the diagnosis result. A diagnosis result display screen W2 illustrated in the drawing is a screen that is subjected to display processing based on the diagnosis result when the diagnosis result is determined by the diagnosis unit 12 and displays the diagnosis result displayed on the user terminal 2, and includes an evaluation display portion W21 and a progress display diagram W22. The diagnosis result display screen W2 illustrated in FIG. 5 includes a plurality of evaluation display portions W21 related to each evaluation item. The diagnosis result display screen W2 may include an advice display portion that displays advice provided by the advice providing unit 14 and a product information display portion that displays product information provided by the care information providing unit 15.

The evaluation display portion W21 is a screen display that displays a graded evaluation (five-grade evaluation in FIG. 5) for any evaluation item included in the diagnosis result. In the example illustrated in FIG. 5, in order to facilitate intuitive understanding, the evaluation display portion W21 performs display in a graph format in which a bar graph that has been lightly colored is colored in another color such as a dark color according to the evaluation, but may display the graded evaluation as a numerical value. The evaluation display portion W21 further includes a reference display that is a screen display indicating an evaluation serving as a reference in a predetermined evaluation item, and can show the evaluation serving as the reference in the predetermined evaluation item (in a case where the evaluation item is the fineness of hair, the fineness of the 4th grade among 5 grades is an evaluation serving as a reference). In this case, the display processing unit 17 displays the reference for each evaluation item based on the reference information on the evaluation serving as the reference for each evaluation item stored in the storage unit 102. A reference for an evaluation may be an average of evaluations for each evaluation item of the user or a value of an evaluation serving as a reference for each evaluation item set depending on the age or gender.

The progress display diagram W22 is a screen display for displaying a transition of the evaluation included in the diagnosis result of the user, and is a screen display showing the transition of the evaluation in the result of the current diagnosis and the result of the past diagnosis up to the previous diagnosis, which is subjected to the display processing based on the past diagnosis result (history information) of the user stored in association with the diagnosis result by the currently executed diagnosis and the user information. In the present embodiment, the progress display diagram is a graph generated for each evaluation item (in the example illustrated in FIG. 5, a graph of a progress related to the fineness of hair), but may be a graph in which transitions of evaluations of the plurality of evaluation items are integrated into one. In the present embodiment, the progress display diagram W22 is shown by a line graph and a bar graph, but may be a graph of any format as long as the graph can display the transition of the evaluation. The progress display diagram W22 illustrated in FIG. 5 is a graph whose vertical axis indicates the evaluations and horizontal axis indicates the number of times of diagnosis, and which indicates a transition of the evaluation in the performed diagnosis, but may be a graph whose horizontal axis indicates time, and which indicates a transition of the evaluations over time.

In the diagnosis result display screen W2, a date may be input by pressing a date display such as a calendar, and the display processing unit 17 may perform display processing on a screen indicating a result of diagnosis on a designated date based on the history information. In this case, based on the history information, the display processing unit 17 may perform display processing on a history display screen indicating the evaluation in the past diagnosis, which has the same screen configuration as the diagnosis result display screen as illustrated in FIG. 5.

### Processing Flow

Hereinafter, a processing flow of the beauty and health information providing device 1 according to the present embodiment will be described.

### Learning Processing

Hereinafter, a processing flow related to learning of the diagnosis model will be described. The data processing unit 13 acquires the inquiry information, the captured image, and diagnosis information, which are raw data before processing stored in the storage unit 102, and performs preprocessing for conversion into a form easy to handle in the diagnosis model. When the learning unit 11 acquires the inquiry information, the captured image, and the diagnosis information, which are processed data preprocessed by the data processing unit 13, the learning unit 11 performs learning of the diagnosis model using the above data as teaching data.

### Diagnosis Processing

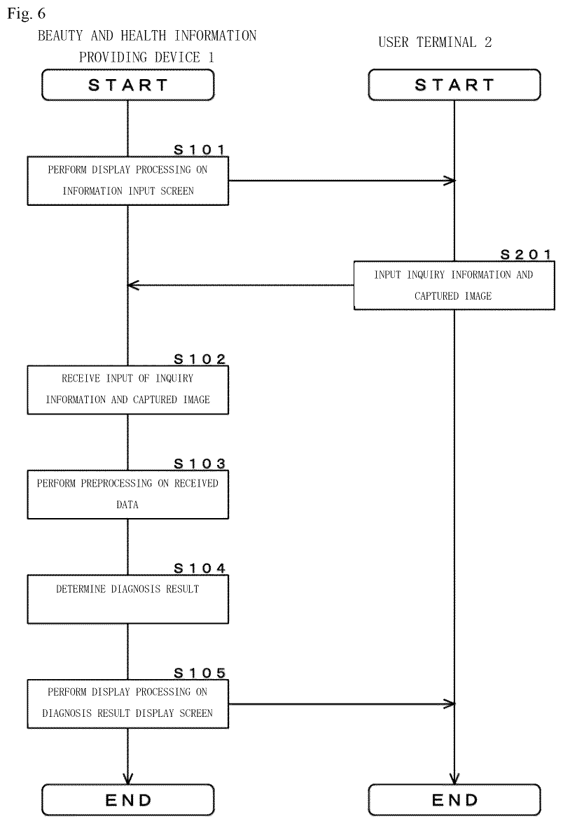

Processing of making a diagnosis of the scalp and/or hair will be described with reference to FIG. 6. When starting diagnosis is selected by the user and the reception unit 16 receives a display request of the information input screen from the user terminal 2, the display processing unit 17 performs display processing of the information input screen (S101). The user terminal 2 receives the input of the inquiry information and the captured image by the user and transmits them to the beauty and health information providing device 1 (S201). The reception unit 16 receives the inquiry information and the captured image from the user terminal 2 (S102). In this case, the beauty and health information providing device 1 may determine whether the received captured image is an image suitable for processing such as diagnosis in the diagnosis unit 12, such as whether the received captured image is an image at a suitable angle, and if the result of the determination is that the received captured image is not a suitable image, the display processing unit 17 may display a warning message or an instruction to transmit a captured image again, and the reception unit 16 may receive an input again. The data processing unit 13 performs preprocessing for converting the raw data such as the inquiry information and the captured image received by the reception unit 16 into a form that is easy to handle in the system (S103). When receiving the inquiry information and the captured image processed by the data processing unit 13, the diagnosis unit 12 determines a diagnosis result based on the inquiry information and the captured image using the diagnosis model (S104). The diagnosis unit 12 determines whether a diagnosis result associated with the user information of the user to be diagnosed is stored in the storage unit 102, and in a case where the user has been diagnosed in the past and there is a past diagnosis result (history information) associated with the user information, the diagnosis unit 12 determines the diagnosis result further based on the history information that is a history of the past diagnosis result of the user in addition to the inquiry information and the captured image. The display processing unit 17 performs display processing of a diagnosis result display screen that displays graded evaluations of a plurality of evaluation items based on the diagnosis result by the diagnosis unit 12 (S105).

After the processing by the diagnosis unit 12 as illustrated in FIG. 6, either or both of the processing of providing the product information by the care information providing unit 15 and the processing of providing the advice by the advice providing unit 14 may be performed.

In the present embodiment, the diagnosis unit 12 determines a graded evaluation of the scalp and/or hair as a diagnosis result based on the inquiry information and the captured image, and provides the graded evaluation as the diagnosis result, but may further provide, for example, a diagnosis result related to a level of well-being, mental health, or the like based on the inquiry result as a diagnosis result. The diagnosis unit 12 may further receive data such as an analysis result of a sample of the scalp, hair, or the like of the user and use the data for determining the diagnosis result. The diagnosis result may include evaluations of items such as hormone balance and a stress value.

### Embodiment 2

In Embodiment 2, an example in which the beauty and health information providing system 0 functions as a system for providing a market place in which a product for maintaining beauty or health can be purchased will be described. In the following example, the care information provided to the user is product information on a product used for beauty and health care.

In the present embodiment, the reception unit 16 receives registration of product information and stores product information in the storage unit 102. The product information is information on a product provided to the user, includes a product name, a product price, a product category, a product image, and a description of the product, and is uniquely identified and managed by identification information such as a product ID. In the present embodiment, the product information is registered by an administrator of the beauty and health information providing system 0, but may be registered by a manufacturer or the like who produces the product. The display processing unit 17 performs display processing on a product purchase screen for describing the product based on the product information and receiving a purchase of the product. In this case, the beauty and health information providing device 1 receives a purchase of the product from the user and performs processing for the purchase such as payment processing. A purchase history of the product of the user in the market place and the information on the product registered as a favorite by the user (favorite registration information) are stored in the storage unit 102 in association with the user information using the identification information such as the user ID as an action history of the user in the market place. The action history of the user may be used for processing related to provision of information on a suitable product by the care information providing unit 15 to be described later, such as being used for collaborative filtering.

The reception unit 16 further receives registration of provision attribute information which is information on an attribute of a user to whom the product information on a product is provided, and stores the provision attribute information in the storage unit 102. The provision attribute information is information on an attribute of a user who receives the provision of the product information on the product, such as to what kind of user the product information is to be provided, and is registered by an administrator of the beauty and health information providing system 0, a manufacturer of the product, or the like. In the present embodiment, the provision attribute information includes information on an answer to the question in the inquiry (for example, the trouble is gray hair), an evaluation value included in the diagnosis result (for example, the evaluation of the moisture content of hair is 4 or more), an environment around the user (for example, a quantity of ultraviolet ray, weather, season, and the like in a living area of the user), a component necessary for the user, and the like. In the present embodiment, the provision attribute information is information on an item corresponding to any information on the user including the user information, the diagnosis result, and the inquiry information, but may include information on the action history of the user such as not purchasing a product (for example, shampoo or the like) of the same category for one month or longer. The product information and the provision attribute information are stored in association with unique identification information such as a product ID. The storage unit 102 may store, as the provision attribute information, a coordinate, a vector, a feature, and the like in a feature space generated based on information on the attribute of the user to whom the product information is to be provided, such as an answer to a question in the inquiry received by the reception unit 16, an evaluation value included in the diagnosis result, and an environment around the user.

The care information providing unit 15 determines the product whose product information is to be provided based on condition information and the information on the user such as the diagnosis result, the inquiry information, and the user information. The care information providing unit 15 refers to the product information stored in the storage unit 102 on the product whose information is determined to be provided, and provides the product information suitable for the user. The condition information is information on a condition on which the information on the product is provided, and is generated based on the product information and the provision attribute information on the attribute of the user who receives the provision of the product information in the present embodiment. Based on the condition information generated based on the product information and the provision attribute information, for example, for a user evaluated as having a small moisture content as a diagnosis result, the care information providing unit 15 determines, for example, a product (for example, shampoo and treatment) set to be provided to a user having a small moisture content in the provision attribute information as a product whose information is to be provided. In this case, based on a plurality of conditions included in the condition information, the care information providing unit 15 may determine a product that satisfies all the conditions, or may determine a product that satisfies a certain condition or more among some conditions, as a product whose information is to be provided. a condition related to a different item may be set for each product, such as that a condition related to the age may be set for a certain product and a condition related to the gender may be set for a certain product.

In the present embodiment, the care information providing unit 15 determines a product having a certain degree or more of relevance to the user as a product whose information is to be provided, based on the condition information and the information on the user including the diagnosis result, the inquiry information, and the user information. In this case, the care information providing unit 15 may determine the product having the highest relevance as a product whose information is to be provided, or may determine a plurality of products having a certain degree or more of relevance as products whose information is to be provided. The care information providing unit 15 may determine a component necessary for the user from the trouble of the user or the evaluation in the diagnosis based on the inquiry information and the diagnosis result, and determine a product including a product having the determined component as a product whose information is to be provided.

The care information providing unit 15 may generate a graph related to a relation of the product based on the product information and the provision attribute information, and use the graph as the condition information for providing the product information. The graph referred to here is not a diagram such as a pie graph or a bar graph, but is a data structure including relations between nodes and nodes, and is a data structure capable of indicating the relations between nodes (people, things, or the like). In the present embodiment, the care information providing unit 15 expresses a relation of a product in a feature space as a graph (for example, coordinates in the feature space such as (X, Y, Z)) based on the product information and the provision attribute information, and determines the product having the closest relation to the user as the product whose information is to be provided. In this case, the care information providing unit 15 may obtain the relevance by referring to a distance between the products as a vector. The storage unit 102 may store graph information (for example, the coordinates in the feature space such as (X, Y, Z)) generated based on the product information and the provision attribute information as the condition information in association with the product information, and the care information providing unit 15 may determine a product whose information is to be provided using the condition information. In this case, the care information providing unit 15 searches the feature space indicating the relations between products based on the information on the user such as the user information, the diagnosis result, and the inquiry information, and determines a product with coordinates closest to or within a certain range from coordinates indicating features of the user as the product whose product information is to be provided. The care information providing unit 15 may determine the product whose information is to be provided by using a model that handles graph data, such as a graph neural network.

The display processing unit 17 may display information on a product determined to be provided by the care information providing unit 15 on the diagnosis result display screen W2 as illustrated in FIG. 5, or may display information on a product to be provided as a recommended product on a market screen for purchasing a product. In order to provide the product information, the reception unit 16 may receive information on a living area of the user such as an address or a region (for example, a prefecture name or “Kanto”) and store the information in the storage unit 102 as the user information. In this case, the care information providing unit 15 accesses an external weather forecast system or the like, acquires information on the environment of the living area of the user, such as the weather and temperature of the living area of the user, based on the information on the living area of the user, such as the address of the user, and uses the acquired information for processing of determining a product whose information is to be provided.

The care information providing unit 15 may obtain the relevance between the users based on the action history of the user in the market place such as the purchase history of the user, determine a product that was purchased or liked by a similar user having a high relevance as a product whose information is to be provided based on the relevance between the users, and provide the product information.

In the present embodiment, the beauty and health information providing system 0 provides information on a suitable product to the user every month. When data to be used for providing the product information is insufficient, such as when the user is not performing diagnosis this month, the display processing unit 17 may perform display processing on a screen for displaying a message prompting the user to perform diagnosis, or may perform display processing on a screen for receiving input of insufficient information.

The reception unit 16 further receives, from a user who has purchased a product or a user who has received provision of a sample, an input of an answer (questionnaire result) to a questionnaire such as an impression of using the product. The questionnaire result received by the reception unit 16 is stored in the storage unit 102 in association with the product by identification information such as a product ID. The questionnaire may be displayed during the inquiry, and the reception unit 16 may receive the answer to the questionnaire simultaneously with the inquiry information. In this case, the display processing unit 17 may perform display processing on a screen for displaying questions of the inquiry and questions of the questionnaire and receiving the answers to the questions, such as the information input screen W1 illustrated in FIG. 4, and receive an input of the answers (inquiry information, questionnaire result) to the questions.

In the present embodiment, the provision attribute information related to the condition for providing the product information is optimized based on the acquired questionnaire result. The care information providing unit 15 may include a collection unit that collects the questionnaire results acquired for each product. In this case, the collection unit collects the content of the questionnaire and the attribute of the user who answered the questionnaire (the attribute of the user based on the diagnosis result, the user information, and the inquiry result). The display processing unit 17 may support optimum setting of the provision attribute information by the user by performing display processing on a collection result of the questionnaire result by the collection unit.

The beauty and health information providing device 1 may include an optimization processing unit for optimizing the provision attribute information. In this case, regarding the optimization of the provision attribute information, the optimization processing unit may perform modeling (for example, modeling the user as coordinates in a graph represented by (X, Y, Z) or the like) on the user who receives the provision of the product information by taking an average of users who purchased the product or users who answered that the product is his or her favorite in the questionnaire, based on information (user information, diagnosis results, inquiry information, and the like) on the users who have actually purchased the product or liked the product, and may perform processing of using the model as the provision attribute information of the product as processing of optimizing the provision attribute information.

### REFERENCE SIGNS LIST

0 beauty and health information providing system
1 beauty and health information providing device
2 user terminal
NW communication network
11 learning unit
12 diagnosis unit
13 data processing unit
14 advice providing unit
15 care information providing unit
16 reception unit
17 display processing unit

1. A beauty and health information providing system that provides a user with beauty and health information on scalp and/or hair, the system comprising:
a storage unit that stores a diagnosis model that performs learning using inquiry information on an answer from a user to an inquiry and a captured image of the scalp and/or hair as input data and using a graded evaluation of the scalp and/or hair as output data;
a reception unit that receives the inquiry information on the answer from the user to the inquiry and the captured image of the scalp and/or hair; and
a diagnosis unit that determines a diagnosis result that is the graded evaluation of the scalp and/or hair based on the inquiry information and the captured image by using the diagnosis model.

2. The beauty and health information providing system according to claim 1, further comprising:
a learning unit, wherein
the learning unit performs the learning of the diagnosis model by using the inquiry information on the answer from the user to the inquiry and the captured image of the scalp and/or hair as the input data and using the graded evaluation of the scalp and/or hair as the output data.

3. The beauty and health information providing system according to claim 1, wherein
the storage unit stores history information that is a past diagnosis result of the user, and
the diagnosis unit determines the diagnosis result further based on the history information.

4. The beauty and health information providing system according to claim 1, further comprising:
a display processing unit, wherein
the diagnosis result includes graded evaluations of a plurality of evaluation items for the scalp and/or hair, and
the display processing unit performs display processing of a diagnosis result display screen that displays the graded evaluations of the plurality of evaluation items for the scalp and/or hair based on the diagnosis result.

5. The beauty and health information providing system according to claim 4, wherein
the diagnosis unit stores history information that is a past diagnosis result of the user in the storage unit in association with a diagnosis date, and
the diagnosis result display screen includes a progress display diagram showing evaluation progress for the scalp and/or hair based on a plurality of the diagnosis results and the diagnosis dates.

6. The beauty and health information providing system according to claim 1, further comprising:
an advice providing unit, wherein
the advice providing unit outputs advice on beauty care of the scalp and/or hair based on the inquiry information.

7. The beauty and health information providing system according to claim 1, further comprising:
a care information providing unit, wherein
the storage unit further stores a plurality of pieces of care information on care to be provided to the user, and
the care information providing unit provides the care information on care suitable for the user based on the diagnosis result and the plurality of pieces of care information.

8. A beauty and health information providing method executable by a computer including a storage unit that stores a diagnosis model that performs learning using inquiry information on an answer from a user to an inquiry and a captured image of scalp and/or hair as input data and using a graded evaluation of the scalp and/or hair as output data, the beauty and health information providing method being for providing information on beauty care of skin and hair to the user, the method comprising:
a reception step of receiving the inquiry information on the answer from the user to the inquiry and the captured image of the scalp and/or hair; and
a diagnosis step of determining a diagnosis result that is the graded evaluation of the scalp and/or hair based on the inquiry information and the captured image by using the diagnosis model.

9. A beauty and health information providing program that provides information on beauty care of skin and hair to a user, the program causing a computer to operate, the computer including
a reception unit that receives inquiry information on an answer from the user to an inquiry and a captured image of scalp and/or hair,
a diagnosis unit that determines a diagnosis result that is a graded evaluation of the scalp and/or hair based on the inquiry information and the captured image by using a diagnosis model, and
a storage unit that stores the diagnosis model that performs learning using the inquiry information on the answer from the user to the inquiry and the captured image of the scalp and/or hair as input data and using the graded evaluation of the scalp and/or hair as output data.
